(19) United States
(12) Patent Application Publication
(10) Pub. No.: US 2020/0129473 A1
(43) Pub. Date: Apr. 30, 2020

(54) USE OF ERIBULIN AND CYCLIN DEPENDENT KINASE INHIBITORS IN THE TREATMENT OF CANCER

(71) Applicant: EISAI R&D MANAGEMENT CO., LTD., Tokyo (JP)

(72) Inventors: Bruce A. LITTLEFIELD, Andover, MA (US); Gary HENDLER, Hatfield (GB)

(21) Appl. No.: 16/631,207

(22) PCT Filed: Jul. 23, 2018
(86) PCT No.: PCT/JP2018/027513

§ 371 Date: Jan. 15, 2020

Related U.S. Application Data
(60) Provisional application No. 62/535,393, filed on Jul. 21, 2017.

(51) Int. Cl. A61K 31/357; A61P 35/00; A61P 35/02; A61K 31/519; A61K 9/00; A61K 45/06

(52) U.S. Cl. CPC A61K 31/357; A61P 35/00; A61P 35/02; A61K 45/06; A61K 9/0019; A61K 9/0053; A61K 31/519

(57) ABSTRACT

The invention features methods for treating and preventing cancer (e.g., an estrogen receptor-positive (ER⁺) breast cancer) in a patient in need thereof by administering eribulin (e.g., eribulin mesylate) in combination with a cyclin dependent kinase (CDK) inhibitor.

### BACKGROUND OF THE INVENTION

Cancer is characterized by the uncontrolled growth of a particular type of cell. It begins in a tissue containing such a cell and, if the cancer has not spread to any additional tissues at the time of diagnosis, may be treated by, for example, surgery, radiation, or another type of localized therapy. However, when there is evidence that cancer has metastasized from its tissue of origin, different approaches to treatment are typically used. Indeed, because it is not possible to determine with certainty the extent of metastasis, systemic approaches to therapy are usually undertaken when any evidence of spread is detected. These approaches can involve the administration of chemotherapeutic drugs that interfere with the growth of rapidly dividing cells, such as cancer cells. Other approaches involve the use of immunotherapy, in which an immune response against cancerous cells in a subject is elicited or enhanced.

Halichondrin B is a structurally complex, macrocyclic compound that was originally isolated from the marine sponge Halichondria okadai, and subsequently was found in Axinella sp., Phakellia carteri, and Lissodendoryx sp. A total synthesis of halichondrin B was published in 1992 (Aicher et al., J. Am. Chem. Soc. 114:3162-3164, 1992). Halichondrin B has been shown to inhibit tubulin polymerization, microtubule assembly, beta^{s}-tubulin crosslinking, GTP and vinblastine binding to tubulin, and tubulin-dependent GTP hydrolysis in vitro. This molecule has also been shown to have anti-cancer properties in vitro and in vivo. Halichondrin B analogs having anti-cancer activities are described in U.S. Pat. No. 6,214,865 B1.

Eribulin is a synthetic analog of halichondrin B. Eribulin is also known as ER-086526, and has been assigned CAS number 253128-41-5 and US NCI designation number NSC-707389. The mesylate salt of eribulin (eribulin mesylate, which is marketed under the trade name HALAVEN® and is also known as E7389) is approved in certain jurisdictions for the treatment of certain patients with breast cancer and for the treatment of certain patients with liposarcoma. In the U.S., for example, HALAVEN® is approved for the treatment of patients with breast cancer who have previously received at least two chemotherapeutic regimens for the treatment of metastatic disease that should have included an anthracycline and a taxane in either the adjuvant or metastatic setting, and for second line liposarcoma treatment.

The chemical name for eribulin mesylate is 11,15:18,21:24,28-triepoxy-7,9-ethano-12,15-methano-9H,15H-furo[3,2-i]furo[2′,3′:5,6]pyrano[4,3-b][I,4]dioxacyclopentacosin-5(4H)-one, 2-[(2,5)-3-amino-2-hydroxypropyl]hexacosahydro-3-methoxy-26-methyl-20,27-bis(methylene)-, (2R,3R,3aS,7R,8aS,9S,I0aR,11S,12R,13aR,13bS,15S,18S,21S,24S,26R,28R,29aS)-methanesulfonate (salt), and it can be depicted as set forth below.

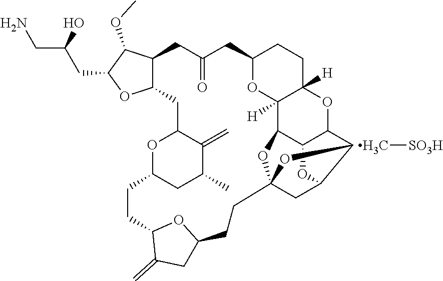

Cancer is typically characterized by disruption(s) in one or more pathways that regulate the cell cycle. Complexes comprised of cyclin-family proteins and cyclin dependent kinases (CDKs), in cooperation with other factors, regulate signaling pathways that control cell proliferation. In many cancers, CDKs are dysregulated, thus making CDKs targets for anti-cancer drug development. There are a number of different classes of CDKs (e.g., CDK1-CDK20) that act at different points in the cell cycle. CDK inhibitors may act selectively on specific CDKs or broadly on a spectrum of CDKs or other enzymes. Palbociclib (PD0332991; Ibrance®) is an example of a selective CDK4 and CDK6 inhibitor. The chemical name of palbociclib is 6-acetyl-8-cyclopentyl-5-methyl-2-[(5-piperazin-1-ylpyridin-2-yl)amino]pyrido[2,3-d]pyrimidin-7-one, and it can be depicted as set forth below.

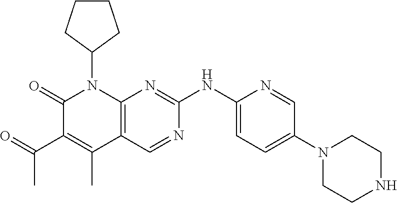 [SMILES: CC(=O)c1c(C)c2cnc(Nc3ccc(N4CCNCC4)cn3)nc2n(C2CCCC2)c1=O]

### SUMMARY OF THE INVENTION

The present invention provides methods of treating and preventing cancer (e.g., estrogen receptor-positive (ER⁺) breast cancer) by administration of eribulin, or a pharmaceutically acceptable salt thereof (e.g., eribulin mesylate), and a CDK inhibitor (e.g., a CDK4/6 inhibitor, such as palbociclib). When the term “eribulin” is used herein, it should be considered as indicating eribulin or a pharmaceutically acceptable salt thereof (such as eribulin mesylate), unless the context indicates otherwise.

The invention provides methods for treating a subject (e.g., a human subject, such as an adult patient or a pediatric patient) having or at risk of developing cancer, the methods including or consisting of administering to the subject (a) eribulin, or a pharmaceutically acceptable salt thereof (e.g., eribulin mesylate), and (b) a cyclin dependent kinase (CDK) inhibitor (e.g., a CDK 4 inhibitor, a CDK 6 inhibitor, or a CDK 4/6 inhibitor; for example, one or more of palbociclib, ribociclib, G1T-28, abemaciclib, and MM-D37K.

In some embodiments, (b) is withheld for a certain period of time during said regimen. For example, (b) may be withheld for one or more days before, during, or after (a) is administered; or (b) may be withheld for two days before, during, or after (a) is administered. Thus, for example, (b) may not be administered within about 24-48 hours before (a), and/or (b) may not be administered within about 24 hours after (a).

In various embodiments, (a) is administered on days 1 and 8 of a 21 day cycle. In some embodiments, (b) is administered on any one or more of days 2-6 (or 7) and 9-13 (or 14) of said 21 day cycle. In other embodiments, (a) is administered on days 1, 8 and 15 of a 28 day cycle. While in further embodiments. (b) is administered on any one or more of days 2-6 (or 7), 9-13 (or 14) and 16-20 (or 21) of said 28 day cycle.

The subject may optionally be diagnosed with cancer, in treatment for cancer, or in post-therapy recovery from cancer. The cancer may be, e.g., a primary tumor, locally advanced, or metastatic. In various examples, the cancer is selected from the group consisting of breast cancer, sarcomas, endometrial cancer, ovarian cancer, prostate cancer, leukemia, lymphoma, lung cancer, neuroendocrine tumors, pheochromocytoma, and thyroid cancer. In certain embodiments, the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor. Furthermore, the cancer may optionally be a hormone responsive cancer (e.g., hormone responsive breast cancer).

In various embodiments, the eribulin or the pharmaceutically acceptable salt thereof is administered by intravenous infusion. The intravenous infusion can optionally be for about 1 to about 20 minutes, e.g., for about 2 to about 5 minutes. In some embodiments, the eribulin or the pharmaceutically acceptable salt thereof is administered in an amount in the range of about 0.1 mg/m² to about 20 mg/m², e.g., 1.1 mg/m² or 1.4 mg/m².

In some embodiments, the CDK inhibitor is administered orally in an amount ranging from 5-350 mg once or twice per day. Thus, for example, when the CDK inhibitor is, e.g., palbociclib, it can be administered in an amount of about 125 mg, 100 mg, 75 mg, 50 mg, or 25 mg per dose.

The methods of the invention optionally have one or more of the following effects, wherein the treating: (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

Also included in the invention are methods for decreasing the size of a tumor in a subject, the methods including or consisting of administering to the subject (a) eribulin, or a pharmaceutically acceptable salt thereof (e.g., eribulin mesylate), and (b) a CDK inhibitor (e.g., palbociclib; also see above and elsewhere herein).

The invention further provides kits for use in treating cancer or decreasing tumor size in a subject, the kit including (a) eribulin, or a pharmaceutically acceptable salt thereof (e.g., eribulin mesylate), and (b) a CDK inhibitor (e.g., palbociclib, ribociclib, G1T-28, abemaciclib, or MM-D37K), optionally in dosage form.

Also included in the invention is eribulin, or a pharmaceutically acceptable salt thereof (e.g., eribulin mesylate), for use in a method for treating a subject having or at risk of developing cancer, the method including administering to the subject (a) eribulin, or pharmaceutically acceptable salt thereof (e.g., eribulin mesylate), and (b) a CDK inhibitor (e.g., palbociclib, ribociclib, G1T-28, abemaciclib, or MM-D37K). Further, the invention includes eribulin (e.g., eribulin mesylate), or a pharmaceutically acceptable salt thereof, for use in a method of making a medicament for treating a subject having or at risk of developing cancer, the method comprising administering to the subject (a) eribulin (e.g., eribulin mesylate), or pharmaceutically acceptable salt thereof, and (b) a CDK inhibitor (e.g., palbociclib, ribociclib, G1T-28, abemaciclib, or MM-D37K).

The methods of the invention provide improved approaches for treating cancer. For example, the combination treatment methods described herein can be used to obtain synergistic effects in which, for example, the effects of the combination are greater than the sum of the effects of the drugs administered individually, as can be determined by those of skill in the art. Additive effects, which are also beneficial, can also be achieved.

Other features and advantages of the invention will be apparent from the following detailed description, drawings, and claims.

### BRIEF DESCRIPTION OF DRAWINGS

FIG. 1 is a series of graphs showing in vitro growth inhibitory activity of eribulin and palbociclib alone and in combination against the indicated human breast carcinomas (MCF-7, T-470, and ZR-75-1).

FIG. 2 is a series of graphs showing in vitro growth, inhibitory activity of eribulin and palbociclib alone and in combination against the indicated human breast carcinomas (MDA-MB-134VI, MDA-MB-175VII, and MDA-MB-415).

FIG. 3 is a series of graphs showing in vitro growth inhibitory activity of eribulin and palbociclib alone and in combination against the indicated human breast carcinomas (MDA-MB-231, HCC70, and HCC1806).

FIG. 4 is a series of graphs showing in vitro growth inhibitory activity of eribulin and palbociclib alone and in combination against the indicated human breast carcinomas (BT-549, Hs578t, and MDA-MB-436).

FIG. 5 is a graph showing the effects of 0.1 mg/kg eribulin (IV) and 75 mg/kg palbociclib (PO) on tumor volume in patient-derived xenograft (PDX) mice (OD-BRE-0192). The day numbers indicated along the x-axis are the days after tumor implantation.

FIG. 6 is a graph showing the effects of 0.1 mg/kg eribulin (IV) and 150 mg/kg palbociclib (PO) on tumor volume in PDX mice (OD-BRE-0192). The day numbers indicated along the x-axis are the days after tumor implantation.

FIG. 7 is a graph showing the effects of 0.25 mg/kg eribulin (IV) and 75 mg/kg palbociclib (PO) on tumor volume in PDX mice (OD-BRE-0192). The day numbers indicated along the x-axis are the days after tumor implantation.

FIG. 8 is a graph showing the effects of 0.25 mg/kg eribulin (IV) and 150 mg/kg palbociclib (PO) on tumor volume in PDX mice (OD-BRE-0192). The day numbers indicated along the x-axis are the days after tumor implantation.

FIG. 9 is a graph showing the effects of 0.1 mg/kg eribulin (IV) and 75 mg/kg palbociclib (PO) on tumor volume in patient-derived xenograft (PDX) mice (OD-BRE-0745). The day numbers indicated along the x-axis are the days after tumor implantation.

FIG. 10 is a graph showing the effects of 0.1 mg/kg eribulin (IV) and 150 mg/kg palbociclib (PO) on tumor volume in PDX mice (OD-BRE-0745). The day numbers indicated along the x-axis are the days after tumor implantation.

FIG. 11 is a graph showing the effects of 0.25 mg/kg eribulin (IV) and 75 mg/kg palbociclib (PO) on tumor volume in PDX mice (OD-BRE-0745). The day numbers indicated along the x-axis are the days after tumor implantation.

FIG. 12 is a graph showing the effects of 0.25 mg/kg eribulin (IV) and 150 mg/kg palbociclib (PO) on tumor volume in PDX mice (OD-BRE-0745). The day numbers indicated along the x-axis are the days after tumor implantation.

### DETAILED DESCRIPTION

The invention provides methods for the treatment or prevention of cancer (e.g., estrogen receptor-positive (ER⁺) breast cancer) involving administration of eribulin or a pharmaceutically acceptable salt thereof (e.g., eribulin mesylate) in combination with a cyclin dependent kinase (CDK) inhibitor (e.g., a CDK4/6 inhibitor, such as palbociclib).

Treatment of cancer according to the methods of the invention can (i) reduce the number of cancer cells; (ii) reduce tumor volume; (iii) increase tumor regression rate; (iv) reduce or slow cancer cell infiltration into peripheral organs; (v) reduce or slow tumor metastasis; (vi) reduce or inhibit tumor growth; (vii) prevent or delay occurrence and/or recurrence of the cancer and/or extend disease- or tumor-free survival time; (viii) increase overall survival time; (ix) reduce the frequency of treatment; and/or (x) relieve one or more of symptoms associated with the cancer.

### Pharmaceutical Compositions, Dosage, and Methods

Pharmaceutical compositions including eribulin and/or a CDK inhibitor (e.g., a CDK4/6 inhibitor, such as palbociclib) can be prepared using standard methods known in the art, or can be obtained from commercial sources. Typically, eribulin and the CDK inhibitor used in the invention are included within separate pharmaceutical compositions but they can, optionally, be included within a single composition. Eribulin is typically provided in liquid form, for intravenous administration, while the CDK inhibitor may optionally be formulated, for example, for oral or intravenous formulation, depending upon the inhibitor selected.

Pharmaceutical compositions used in the invention can be prepared by, for example, mixing or dissolving the active ingredient(s), having the desired degree of purity, in a physiologically acceptable diluent, carrier, excipient, or stabilizer (see, e.g., Remington's Pharmaceutical Sciences (22^{nd} edition), ed. A. Gennaro, 2012, Lippincott, Williams & Wilkins, Philadelphia, Pa.). Acceptable diluents include water and saline, optionally including buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counter ions such as sodium; and/or nonionic surfactants such as TWEEN™, PLURONICS™, or PEG.

In preparing compositions for oral dosage form, any of the usual pharmaceutical media can be employed, for example, water, glycols, oils, alcohols, flavoring agents, preservatives, coloring agents. In addition, carriers such as starches, sugars, microcrystalline cellulose, diluents, granulating agents, lubricants, binders, disintegrating agents, and the like can be used in the case of oral solid preparations such as, for example, powders, capsules, and tablets.

Optionally, the formulations of the invention contain a pharmaceutically acceptable preservative. In some embodiments the preservative concentration ranges from 0.1 to 2.0%, typically v/v. Suitable preservatives include those known in the pharmaceutical arts, such as benzyl alcohol, phenol, m-cresol, methylparaben, and propylparaben. Furthermore, the eribulin and/or the CDK inhibitor formulations can optionally include a pharmaceutically acceptable salt, such as sodium chloride at, for example, about physiological concentrations. Thus, in one example, eribulin (e.g., eribulin mesylate) is formulated in 0.9% Sodium Chloride Injection (USP).

The formulations noted above (and others) can be used for administration of the drugs. Thus, the drugs can be administered by routes including intravenous, intra-tumoral, peri-tumoral, intra-arterial, intra-dermal, intra-vesical, ophthalmic, intramuscular, intradermal, intraperitoneal, pulmonary, subcutaneous, and transcutaneous routes. Other routes can also be used including, for example, oral, transmucosal, transdermal, inhalation, intravaginal, and rectal administration routes.

The dosage of eribulin and the CDK inhibitors described herein administered can differ markedly depending on the type of target disease, the choice of delivery method, as well as the age, sex, and weight of the patient, the severity of the symptoms, along with other factors. The dosage to use can be determined by those of skill in the art based on factors such as these. Eribulin and examples of CDK inhibitors that can be used in the methods of the invention, as well as administration regimens, are described further below.

### Eribulin

Methods for the synthesis of eribulin are described, for example, in U.S. Pat. Nos. 6,214,865; 7,982,060; 8,350,067; and 8,093,410, each of which is incorporated herein by reference. As noted above, eribulin mesylate is available commercially and is marketed as HALAVEN®.

Eribulin can optionally be used in the present invention in salt forms. There are no particular limitations as to the salt used, whether inorganic acid salt or organic acid salt. For example, the salt can be selected from mesylic acid salt (e.g., eribulin mesylate), hydrochloric acid salt, sulfuric acid salt, citrate, hydrobromic acid salt, hydroiodine acid salt, nitric acid salt, bisulfate, phosphoric acid salt, super phosphoric acid salt, isonicotinic acid salt, acetic acid salt, lactic acid salt, salicic acid salt, tartaric acid salt, pantotenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate), and so on. Moreover, it is acceptable to use salt of aluminum, calcium, lithium, magnesium, sodium, zinc, and diethanolamine.

The daily dosage of eribulin (e.g., eribulin mesylate) can be in the range of, e.g., 0.001 mg/m² to about 100 mg/m² (e.g., in the range of about 0.1 mg/m² to about 50 mg/m² or in the range of about 0.7 mg/m² to about 1.5 mg/m², or in any single amount within these ranges (e.g., 1.4 mg/m² or 1.1 mg/m²)). Eribulin can be administered as a single dose once per day, week, month, or year, or more than one dose of eribulin can be administered per day, week, month, or year. For example, in one administration protocol, eribulin can be administered once on days 1 and 8 of a 21-day cycle. More specifically, a recommended dose of eribulin mesylate is 1.4 mg/m² administered intravenously over 2 to 5 minutes on days 1 and 8 of a 21-day cycle. A recommended dose of eribulin mesylate in patients with mild hepatic impairment (Child-Pugh A) is 1.1 mg/m² administered intravenously over 2 to 5 minutes on days 1 and 8 of a 21-day cycle, while a recommended dose of eribulin mesylate in patients with moderate hepatic impairment (Child-Pugh B) is 0.7 mg/m² administered intravenously over 2 to 5 minutes on days 1 and 8 of a 21-day cycle. Further, a recommended dose of eribulin mesylate in patients with moderate renal impairment (creatinine clearance of 30-50 mL/min) is 1.1 mg/m² administered intravenously over 2 to 5 minutes on days 1 and 8 of a 21-day cycle. These or other lower doses of eribulin mesylate can optionally be, used in the context of combination treatment, according to the methods of the present invention.

### CDK Inhibitors

CDK inhibitors that can be used in the invention can be specific for CDKs 4 and/or 6, or can be active against one, more than one, or all classes of CDKs. In various examples, the CDK inhibitor used in the invention is a CDK 4/6 dual inhibitor. CDK inhibitors can be classified based on their specificity or, alternatively, based on their mechanism of action or chemical structure. For example, CDK inhibitors that can be used in the invention can be classified based on their mechanism of action, as ATP-competitive inhibitors (e.g., molecules that bind the ATP pocket of a CDK) or ATP non-competitive inhibitors (e.g., substrate-competitive CDK inhibitors, protein-protein interface CDK inhibitors, allosteric CDK inhibitors, covalent CDK inhibitors, or peptidomimetics). Additionally, CDK inhibitors that can be used in the invention can optionally fall within one of the following classes, based on their chemical structures: alkaloid-derivatives (e.g., staurosporine and 7-hydroxystaurosporine [UCN01]), aminoacridines, flavonoid derivatives (flavopiroidol [Alvocidib]), flavone derivatives, indole derivatives, indolinone derivatives, paullone derivatives, purine derivatives, pyrimidine derivatives, pyridine derivatives, pyrazole derivatives, thiazole derivatives, and triazole derivatives.

In one specific example, the CDK inhibitor used in the invention is palbociclib, which is a selective CDK 4 and CDK 6 inhibitor. Methods for the synthesis of palbociclib are described, for example, in U.S. Pat. Nos. 6,936,612 and 7,781,583, each of which is incorporated herein by reference. Palbociclib can be administered as a single dose once per day, week, month, or year, or more than one dose of palbociclib can be administered per day, week, month, or year, optionally by the oral route. For example, in one administration protocol, palbociclib can be administered (e.g., by the oral route) once daily for 21 consecutive days followed by 7 days off treatment. In other examples, palbociclib is administered on a daily, semi-weekly, 5 days per week, weekly, bi-weekly, or monthly basis. Each dose of palbociclib can be, for example, 5-350 mg (e.g., 5, 25, 30, 35, 40, 45, 50, 75, 100, 125, 150, 200, 250, 300, or 350 mg), administered optionally by the oral route.

In other specific examples, the CDK inhibitor used in the invention is a CDK 4/6 inhibitor selected from the group consisting of ribociclib, G1T-28, abemaciclib, and MM-D37K, which can be administered as determined to be appropriate by those of skill in the art. Thus, for example, ribociclib can optionally be administered in an amount ranging from 200-1000 mg/day (e.g., 400-800, 500-700, or 600 mg/day), while abemaciclib can optionally be administered in an amount ranging from 50-500 mg/day (e.g., 100-400 or 200-300 mg/day). When palbociclib is indicated herein to be a CDK inhibitor that can be used in the invention, it is to be understood that any one of ribociclib, G1T-28, abemaciclib, and MM-D37K can be used in its place.

### Combination Administration Regimens

As noted above, according to the methods of the invention, eribulin (e.g., eribulin mesylate) and a CDK inhibitor (e.g., a CDK4/6 inhibitor, such as palbociclib) are administered in combination. In some embodiments, eribulin (e.g., eribulin mesylate) and the CDK inhibitor (e.g., a CDK4/6 inhibitor, such as palbociclib) are administered substantially simultaneously. In some embodiments, eribulin (e.g., eribulin mesylate) and the CDK inhibitor (e.g., a CDK4/6 inhibitor, such as palbociclib) are administered separately, e.g., eribulin is administered first, followed by administration of the CDK inhibitor; or CDK inhibitor is administered first, followed by administration of the eribulin. In some embodiments, eribulin (e.g., eribulin mesylate) and the CDK inhibitor (e.g., a CDK4/6 inhibitor, such as palbociclib) are administered substantially simultaneously, followed by administration of eribulin or the CDK inhibitor. In some embodiments, eribulin (e.g., eribulin mesylate) or the CDK inhibitor (e.g., a CDK4/6 inhibitor, such as palbociclib) is administered first, followed by administration of eribulin and the CDK inhibitor substantially simultaneously. The administrations can begin on the same day or treatment using one agent can start, e.g., 1, 2, 3, 4, 5, or 6 weeks before treatment the other, as can be determined to be appropriate by those of skill in the art.

In various examples, eribulin and the CDK inhibitor (e.g., a CDK4/6 inhibitor, such as palbociclib) are administered according to a regimen including multiple days (e.g., one or more cycles of, e.g., 21 or 28 days). Typically, eribulin is administered such that once it is administered on a particular day, it is not administered again for several days (e.g., 4, 5, 6, or 7 days, or 1, 2, or 3 weeks). For example, an approved dosing regimen for eribulin is on days 1 and 8 of a 21 day cycle. Optionally, one of the drugs is withheld for a certain period of time (e.g., a certain number of days) within a cycle. For example, the CDK inhibitor can be withheld for a certain period of time (e.g., one, two, three, four, or more days) during the regimen. In further detail, the CDK inhibitor can optionally be withheld, e.g., for one or more (e.g., two, three, four, or more) days before, during, and/or after eribulin administration. Thus, the CDK inhibitor can be withheld on the day that eribulin is administered, as well as for one or more (e.g., two, three, four, or more) days before and/or after eribulin is administered.

In specific examples, the CDK inhibitor (e.g., CDK4/6 inhibitor, such as palbociclib) is not administered within about 24-48 hours before the eribulin and/or is not administered within about 24 hours after the eribulin. Thus, for example, eribulin may be administered on days 1 and 8 of a 21 day cycle, and the CDK inhibitor (e.g., a CDK4/6 inhibitor, such as palbociclib) can be administered on any one or more of days 2-6 (or 7) and 9-13 (or 14) of this cycle, as determined to be appropriate by those of skill in the art. In another example, eribulin may be administered on days 1, 8 and 15 of a 28 day cycle, and the CDK inhibitor (e.g., a CDK4/6 inhibitor, such as palbociclib) can be administered on any one or more of days 2-6 (or 7), 9-13 (or 14) and 16-20 (or 21) of this cycle, as determined to be appropriate by those of skill in the art.

In the combination regimens described above, the eribulin can be administered at a standard daily dosage of 1.4 mg/m² by, e.g., intravenous infusion over 2 to 5 minutes. Alternatively, the dosage of the eribulin can be reduced to, e.g., 1.1 mg/m², 0.9 mg/m², or 0.7 mg/m², as determined to be appropriate by one of skill in the art due to factors such as, for example, the development of neutropenia. In a regimen in which eribulin and a CDK inhibitor are each administered on the same day 1 of a cycle, it may be desirable to have the daily initial eribulin dosage be reduced (e.g., 1.1 mg/m²). Further reduction can be considered, for example, in the event of the development of neutropenia. The reduced dosages can also be administered by intravenous infusion, optionally over 2-5 minutes.

The amount of CDK inhibitor (e.g., a CDK4/6 inhibitor, such as palbociclib) used in the combination regimens described above can be as described above (e.g., for palbociclib, 5-350 mg per oral dose, such as 125 mg per oral dose). Alternatively, the amount of CDK inhibitor (e.g., a CDK4/6 inhibitor, such as palbociclib) administered can be reduced (e.g., for palbociclib, reduced to 100, 75, 50, or 25 mg per oral dose), as determined to be appropriate by one of skill in the art.

In addition to eribulin and one or more CDK inhibitors (e.g., a CDK4/6 inhibitor, such as palbociclib), the methods of the present invention can also include the administration of one or more additional therapeutic agents. Among these agents, anti-hormonal agents (e.g., fulvestrant, tamoxifen, toremifene, or aromatase inhibitors (e.g., letrozole)), immunomodulatory agents (e.g., antibodies or vaccines), chemotherapeutic/antitumor agents, antibacterial agents, anti-emetics, and anti-inflammatory agents are suitable. In the case of fulvestrant, for example, the additional therapeutic agent can be administered, for example, in the amount of 500 mg i.m. into the gluteal area as two 5 mL injections on days 1, 15, and 29, and once monthly thereafter. Alterations of this amount and regimen can be used in the invention, as determined to be appropriate by those of skill in the art.

In other instances, eribulin (e.g., eribulin mesylate) and one or more CDK inhibitors (e.g., a CDK4/6 inhibitor, such as palbociclib, ribociclib, G1T-28, abemaciclib, or MM-D37K) can be used in a treatment regimen as the sole therapeutic (e.g., sole anti-cancer) agents. Thus, the methods of the invention can consist of administration of (a) eribulin or a pharmaceutically acceptable salt thereof (e.g., eribulin mesylate), and (b) a CDK inhibitor (e.g., a CDK4/6 inhibitor, such as palbociclib).

### Cancers

The methods of the invention can be used to treat (including, e.g., delay progression) or prevent cancer (e.g., ER⁺breast cancer, such as ER⁺/human epidermal growth factor receptor 2 negative (HER2⁻) breast cancer) in a subject (e.g., a human patient) and/or to decrease tumor size. An ER⁺breast cancer is characterized by cancer cells that have estrogen receptors inside or on the cells and are typically stimulated to proliferate in response to estrogen. The subject can be diagnosed with cancer (e.g., ER⁺breast cancer), at risk for developing cancer, in treatment for cancer, or in post-therapy recovery from cancer. Further, the methods can be used to treat or prevent metastases and/or recurrence. The treatment can be chemotherapeutic alone, although treatment in combination with a surgical procedure to remove or reduce the size of a tumor (e.g., neo-adjuvant treatment), radiation therapy, anti-hormonal, immunotherapy, and/or ablation therapy is also included in the invention. The cancer may be a primary tumor, locally advanced, or metastatic, and optionally may be hormone responsive.

Thus, the methods of the invention can be used to treat or prevent cancer such as, for example, breast cancer, sarcomas, endometrial cancer, ovarian cancer, prostate cancer, leukemia, lymphoma, lung cancer, neuroendocrine tumors, pheochromocytoma, hepatocellular carcinoma and thyroid cancer.

Specifically in regard to breast cancer, the methods of the invention can be used to treat or prevent, e.g., estrogen receptor-positive breast cancer, estrogen receptor-positive/HER2-negative breast cancer, triple-positive breast cancer, HER2-negative breast cancer, triple-negative breast cancer, HER2-positive breast cancer, estrogen receptor-negative breast cancer, progesterone receptor-positive breast cancer, and progesterone receptor-negative breast cancer. The breast cancer further may be ductal carcinoma in situ (DCIS), invasive ductal carcinoma, invasive lobular carcinoma, locally advanced breast cancer, metastatic breast cancer, inflammatory breast cancer, Paget disease of the nipple, or phyllodes tumor. Of particular note is estrogen receptor-positive breast cancer including, for example, estrogen receptor-positive/HER2-negative breast cancer.

Patients'that can be treated according to the methods of the invention include adults (e.g., people older than 18 or 21 years of age), as well as pediatric patients (e.g., patients up to and including the age of 18 or 21 years of age), who have cancer, e.g., a cancer type listed herein. In regard to pediatric patients, specific examples of cancers that can be treated include, e.g., sarcomas and leukemias, such as those listed above.

### Kits

The invention also provides kits that include a container with eribulin (e.g., eribulin mesylate) and/or a container with a CDK inhibitor described herein (e.g., palbociclib; also see above). The eribulin and/or the CDK inhibitor in such kits can be provided in amounts sufficient to treat cancer (e.g., an ER⁺breast cancer; see, e.g., the lists set forth above) in a patient in need thereof (e.g., amounts sufficient for a single administration or for multiple administrations). The kits can thus include multiple containers that each include effective amounts of single-dose eribulin and/or the CDK inhibitor pharmaceutical composition(s). Optionally, instruments and/or devices necessary for administering the pharmaceutical composition(s) can also be included in the kits. Furthermore, the kits can include additional components, such as instructions or administration schedules, for treating a patient with cancer (e.g., an ER⁺breast cancer) with the eribulin and/or the CDK inhibitor described herein.

The present invention is illustrated by the following examples, which are in no way intended to be limiting of the invention.

### EXAMPLES

The growth inhibitory activities of two test agents, eribulin and palbociclib, were determined alone and in combination in cell line-based assays (Example 1) and in patient-derived xenograft (PDX) animal models (Example 2).

### Example 1

Eribulin and palbociclib, alone and in combination, were tested against 12 human breast cancer cell lines (Table 1). The human breast tumor cell lines were selected based on their profile characteristics of being either: Her2⁻ and estrogen receptor positive (ER⁺), or Triple Negative (Her2⁻, ER⁻, and progesterone receptor negative [PR⁻]).

TABLE 1
Cell Lines Summary

| Cell line | Histotype | Profile Status |
|---|---|---|
| MCF-7 | Human Mammary | Her2⁻ and ER⁺ |
| | Gland Adenocarcinoma | |
| T-47D | Human Mammary Ductal | Her2⁻ and ER⁺ |
| | Carcinoma from | |
| | Metastatic Site: Pleural Effusion | |
| ZR-75-1 | Human Mammary Ductal | Her2⁻ and ER⁺ |
| | Carcinoma | |
| MDA-MB-134VI | Human Breast Ductal | Her2⁻ and ER⁺ |
| | Carcinoma | |
| MDA-MB-175VII | Human Mammary Gland | Her2⁻ and ER⁺ |
| | Ductal Carcinoma | |
| MDA-MB-415 | Human Mammary gland/breast; | Her2⁻ and ER⁺ |
| | derived from metastatic site | |
| MDA-MB-231 | Human Mammary Gland | Triple Negative |
| | Adenocarcinoma | |
| HCC70 | Human Breast Ductal Carcinoma | Triple Negative |
| HCC1806 | Human Squamous Carcinoma | Triple Negative |
| BT-549 | Human Breast Carcinoma | Triple Negative |
| Hs578t | Human Breast Carcinoma | Triple Negative |
| MDA-MB-436 | Human Adenocarcinoma | Triple Negative |
| | Derived from Metastatic Site | |

### Materials and Methods

### Cell Culture

MCF-7 human breast cancer cells were cultured in RPMI 1640 medium supplemented with 10% fetal bovine serum (FBS), 2 mM glutamine, 10 mM 4-(2-hydroxyethyl)-1-piperazineethanesulfonic acid (HEPES), 0.075% sodium bicarbonate, 100 units/mL sodium penicillin G, 100 μg/mL streptomycin sulfate and 25 μg/mL gentamicin. T-47D human breast cancer cells were cultured in RPMI 1640 medium supplemented with 10% FBS, 4.5 g/L glucose, 2 mM glutamine, 10 mM HEPES, 0.2 units/mL Insulin, 1 mM sodium pyruvate, 100 units/mL sodium penicillin G, 100 μg/mL streptomycin sulfate and 25 μg/mL gentamicin. ZR-75-1 human breast cancer cells were cultured in RPMI 1640 medium supplemented with 10% FBS, 4.5 g/L glucose, 2 mM glutamine, 10 mM HEPES, 1 mM sodium pyruvate, 100 units/mL sodium penicillin G, 100 μg/mL streptomycin sulfate and 25 μg/mL gentamicin. MDA-MB-134-VI human breast cancer cells were cultured in Leibovitz's L-15 medium supplemented with 20% FBS, 2 mM glutamine, 100 units/mL sodium penicillin G, 100 μg/mL streptomycin sulfate and 25 μg/mL gentamicin. MDA-MB-175-VII human breast cancer cells were cultured in Leibovitz's L-15 medium supplemented with 10% FBS, 2 mM glutamine, 10 mM HEPES, 100 units/mL sodium penicillin G, 100 μg/mL streptomycin sulfate and 25 μg/mL gentamicin. MDA-MB-415 human mammary gland/breast cancer cells were cultured in Leibovitz's L-15 medium supplemented with 15% FBS, 10 μg/mL human insulin, 10 μg/mL glutathione, 2 mM glutamine, 100 units/mL sodium penicillin G, 100 μg/mL streptomycin sulfate and 25 μg/mL gentamicin. MDA-MB231, HCC1806, BT-549, and Hs578t human breast cancer cells were cultured in RPMI 1640 medium supplemented with 10% FBS, 2 mM glutamine, 100 units/mL sodium penicillin G, 100 μg/mL streptomycin sulfate and 25 μg/mL gentamicin. HCC70 human breast cancer cells were cultured in RPMI 1640 medium supplemented with 10% FBS, 2 mM glutamine, 4.5 g/L glucose, 10 mM HEPES, 0.075% sodium bicarbonate, 1 mM sodium pyruvate, 100 units/mL sodium penicillin G, 100 μg/mL streptomycin sulfate and 25 μg/mL gentamicin. MDA-MB-436 human adenocarcinoma derived cancer cells were cultured in Leibovitz's L-15 medium supplemented with 10% FBS, 10 μg/mL bovine insulin, 16 μg/mL glutathione, 2 mM glutamine, 100 units/mL sodium penicillin G, 100 μg/mL streptomycin sulfate and 25 μg/mL gentamicin. The tumor cells were cultured in tissue culture flasks in a humidified incubator at 37° C., in an atmosphere of 5% CO2 and 95% air; except for the MDA-MB-134-VI, MDA-MB-175-VII, MDA-MB-415, and MDA-MB-436 these cells were cultured at 37° C. in 100% air.

### Therapeutic Agents

Eribulin was supplied as a stock solution (10 mM), and was stored protected from light at −80° C. On Day 1 of the study, the stock was thawed and a 1000× stock (1 mM) was prepared using 10 μL of the stock solution diluted in 90 μL of DMSO. The 1000× stock was used to prepare the 10×(10 μM) drug prep solution in 1% DMSO in media.

Palbociclib was obtained from SelleckChem (Catalog No. S1116). It was supplied as a 10 mM stock solution in DMSO, and stored at −80° C. The 10 mM stock served as the 1000× stock and a 10× stock was prepared by diluting the 10 mM stock diluted in 1% DMSO in media to a concentration of 100 μM.

### Proliferation Determination

The selected human tumor cells were seeded at 2,000-10,000 cells/well in a clear polystyrene 96-well microculture plate (Corning® Costar® 96-well flat bottom plate, Cat. No. 3997) in a total volume of 90 μL per well. After 24 hours of incubation in a humidified incubator at 37° C. with 5% CO₂ and 95% air (except for the MDA-MB-134-VI, MDA-MB-175-VII, MDA-MB-415, and MDA-MB-436, these cells were cultured at 37° C. in 100% air), 10 μL of the 10× drug prep was added. In a first experiment, 10 μL of the 10× Eribulin drug prep solution was added to 90 μL of cells plated in media for a final concentration of 1 μM at the top concentration diluted 1:4 for a total of 10 dilutions (1000, 250, 62.50, 15.62, 3.90, 0.98, 0.24, 0.06, 0.02, and 3.8×10⁻³ nM). For palbociclib, 10 μL of the 10× drug prep solution was added to 90 μL of cells plated in media for a final concentration of 10 μM at the top concentration diluted 1:4 for a total of 10 dilutions (10, 2.5, 0.625, 0.156, 0.039, 9.75×10⁻³, 2.44×10⁻³, 6.09×10⁻⁴, 1.52×10⁻⁴, and 3.81×10⁻⁵ μM). In a second experiment, the potentiation determination, 10 μL, of the 10× drug prep solution was added to 90 μL of cells plated in media for a final concentration of 1 μM at the top concentration diluted 1:4 for a total of 10 dilutions (1000, 250, 62.50, 15.62, 3.90, 0.98 0.24, 0.06, 0.02, and 3.8×10⁻³ nM) for eribulin and a constant dose of palbociclib (10 μM) across all dilutions. An additional potentiation determination was completed as described above with the use of a 1 μM constant dose of palbociclib across all dilutions.

After 72 hours (96 total hours) of culture, the plated cells and Cell Titer-Glo® (Promega #G7571) reagents were brought to room temperature and allowed to equilibrate for 30 minutes. One hundred (100) μL of the Cell Titer-Glo® reagent was added to each well. The plate was shaken for two minutes to induce lysis and then left to equilibrate for ten minutes to stabilize the luminescent signal. The medium/Cell Titer-Glo® reagent was transferred to a white polystyrene 96-well microculture plate (Corning® Costar® 96-well flat bottom plate, Cat. No. 3917) before determining luminescence on the Tecan GENios microplate.

Percent inhibition of cell growth was calculated relative to untreated control wells. All tests were performed in duplicate at each concentration level. The IC₅₀ value for the test agents was estimated using Prism 6.05 by curve-fitting the data using the following four parameter-logistic equation:

$$Y = \frac{Top - Bottom}{1 + ( X / {IC}_{50} )} + Bottom$$

where Top is the maximal % of control luminescence, Bottom is the minimal % of control luminescence at the highest agent concentration, Y is the % of control luminescence, X is the agent concentration, IC₅₀ is the concentration of agent that inhibits cell growth by 50% compared to the control cells, and n is the slope of the curve.

### Statistical and Graphical Analysis

Prism (GraphPad) 6.05 for Windows was used for graphical presentations and statistical analyses.

### Results

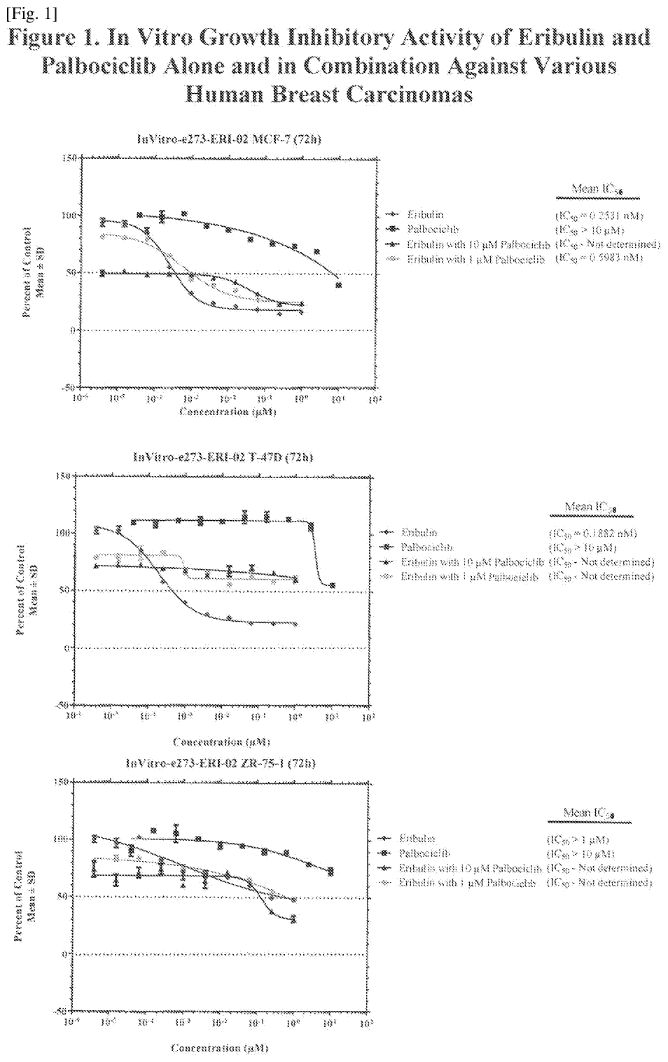

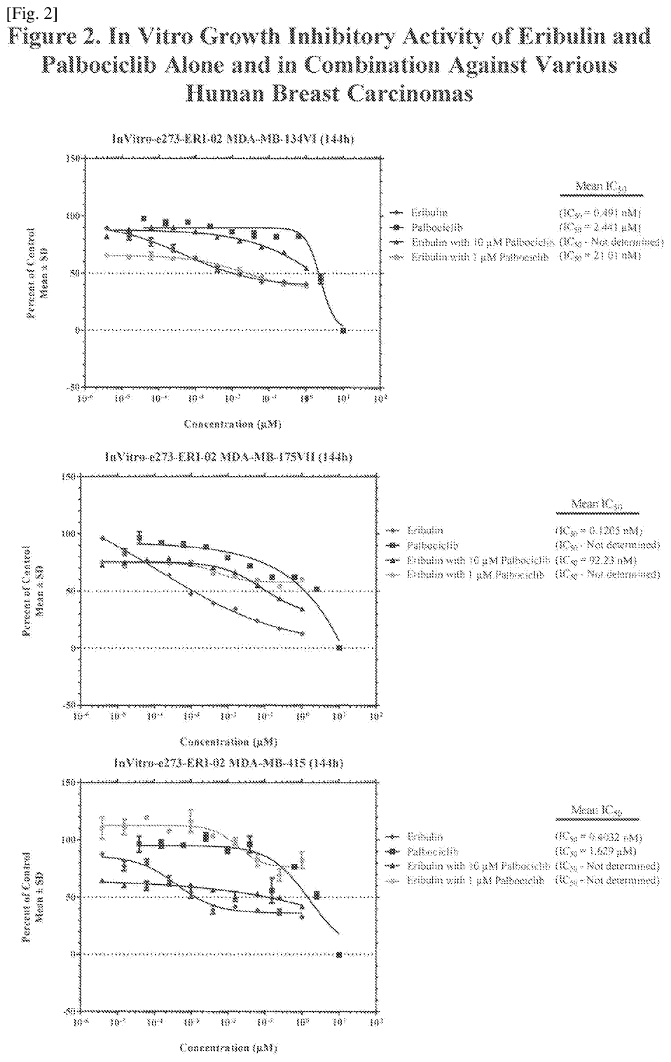

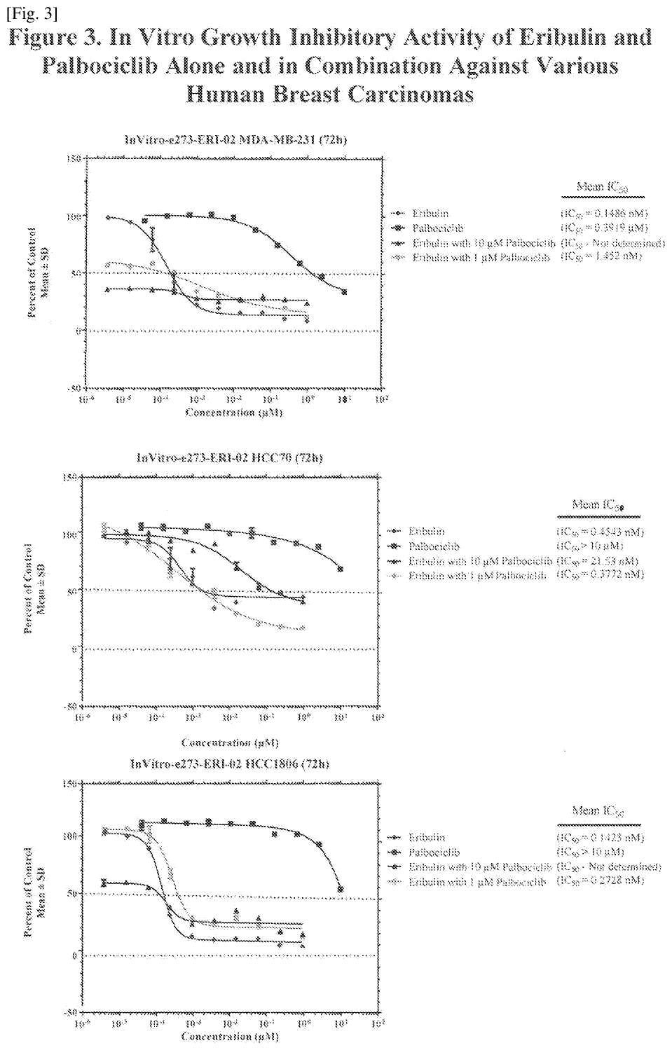

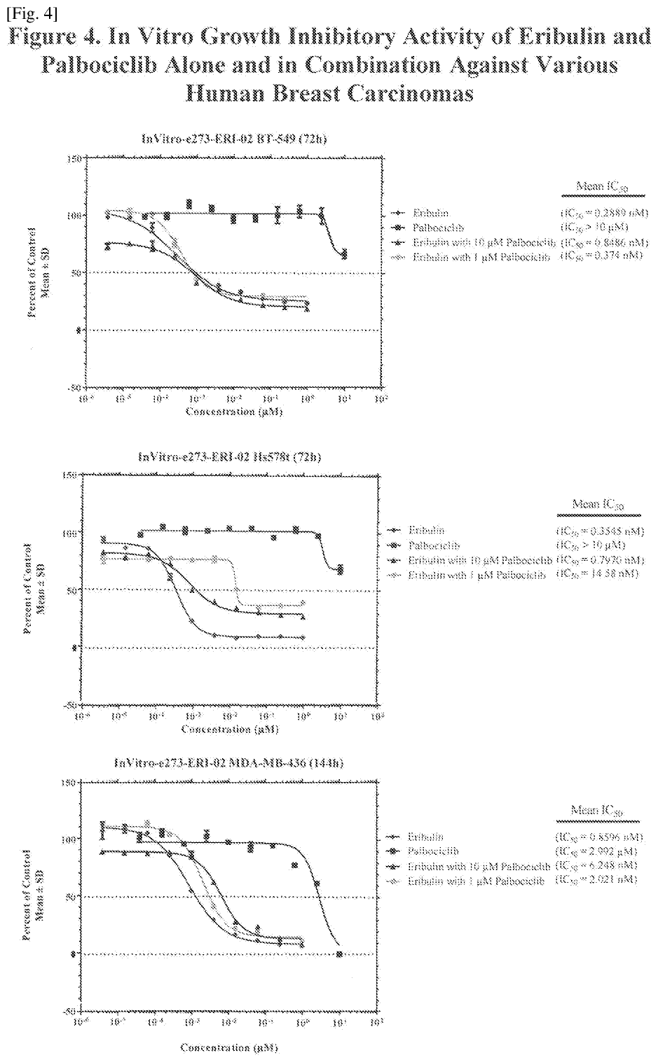

The inhibitory activity of eribulin and palbociclib, alone and in combination, was evaluated against a panel of 12 human breast cancer cell lines as described in Table 1. Results for the first experiment, IC50 determination for single agents, are shown in Table 2 and FIGS. 1-4. Overall, eribulin showed IC50 lower than 1 nM for all cell lines, except for ZR-75-1. Specifically, IC50 values for eribulin were 0.2531 nM for MCF-7, 0.1882 nM for T-47D, >1000 nM for ZR-75-1, 0.4910 nM for MDA-MD-134VI, 0.1205 nM for MDA-MD-175VII, 0.4032 for MDA-MD-415, 0.1486 nM for MDA-MD-231, 0.4543 nM for HCC70, 0.1423 nM for HCC1806, 0.2889 nM for BT549, 0.3545 nM for Hs578t, and 0.8596 nM for MDA-MD-436. On the other hand, palbociclib was much less potent, IC50 values were determined as >10 μM for MCF-7, >10 μM for T-47D, >10 μM for ZR-75-1, 2.441 μM for MDA-MB-134VI, not determined for MDA-MB-175VII, 1.629 μM for MDA-MB-415, 0.3919 μM for MDA-MB-231, >10 μM for HCC70, >10 μM for HCC1806, >10 μM for BT-549, >10 μM for Hs578t, and 2.992 μM for MDA-MB-436.

TABLE 2
Single Agent IC₅₀ Value Determination
Mean IC₅₀ Determination

| | Experiment | | |
|---|---|---|---|
| Cell Line | Number | Eribulin (nM) | Palbociclib (μM) |
| MCF-7 | e003 | 0.2531 | >10 |
| T-47D | e003 | 0.1882 | >10 |
| ZR-75-1 | e003 | >1000 | >10 |
| MDA-M -134VI | e002 | 0.4910 | 2.441 |
| MDA-MB-175VII | e004 | 0.1205 | nd |
| MDA-MB-415 | e004 | 0.4032 | 1.629 |
| MDA-MB-231 | e003 | 0.1486 | 0.3919 |
| HCC70 | e003 | 0.4543 | >10 |
| HCC1806 | e003 | 0.1423 | >10 |
| BT-549 | e003 | 0.2889 | >10 |
| Hs578t | e003 | 0.3545 | >10 |
| MDA-MB-436 | e004 | 0.8596 | 2.992 |

nd, not determined

The potentiation determination was carried out as described in the second experiment. Eribulin was tested at concentrations ranging from 3.8 pM to 1 μM and a constant dose of either 10 μM or 1 μM of palbociclib across all dilutions. Results for IC₅₀ values are shown in Table 3 and FIGS. 1-4. No potentiation effect was observed across all cell lines using a constant dose of 10 μM or 1 μM of palbociclib.

The IC₅₀ values for eribulin with a constant dose of 10 μM of palbociclib were not determined for lines MCF-7, T-47D, ZR-75-1, MDA-MB-134VI, MDA-MB-415, MDA-MB231, and HCC1806; whereas IC₅₀ values were determined as 92.93 nM for MDA-MB-175VII, 21.53 nM for HCC70, 0.8486 nM for BT-549, 0.7970 nM for Hs578t, and 6.248 nM for MDA-MB-436.

The eribulin IC₅₀ values with a constant dose of 1 μM of palbociclib were not determined for cell lines T-47D, ZR-75-1, MDA-MB-175VII, and MDA-MB-415; whereas IC₅₀ values were determined as 0.5983 nM for MCF-7, 21.01 nM for MDA-MB-134VI, 1.452 nM for MDA-MB231, 0.3772 nM for HCC70, 0.2728 nM for HCC1806, 0.3740 nM for BT-549, 14.58 nM for Hs578t, and 2.021 nM for MDA-MB-436.

TABLE 3
Summary of Combination of Eribufin dose response (3.8 pM
to 1 μM) and constant dose (10 or 1 μM) of Palbociclib.
Mean IC₅₀ Determination (nM)

| | With constant 10 μM | With constant 1 μM |
|---|---|---|
| Cell Line | Palbociclib | Palbociclib |
| MCF-7 | nd | 0.5983 |
| T-47D | nd | nd |
| ZR-75-1 | nd | nd |
| MDA-MB-134VI | nd | 21.01 |
| MDA-MB-175VII | 92.23 | nd |
| MDA-MB-415 | nd | nd |
| MDA-MB231 | nd | 1.452 |
| HCC70 | 21.53 | 0.3772 |
| HCC1806 | nd | 0.2728 |
| BT-549 | 0.8486 | 0.3740 |
| Hs578t | 0.7970 | 14.58 |
| MDA-MB-436 | 6.248 | 2.021 |

nd, not determined

### Example 2

We examined combinations of eribulin and palbociclib in two patient-derived xenograft (PDX) models of HR+/Her2−breast cancers grown in immunosuppressed mice. Using dose levels that elicited only minimal antitumor effects when each agent was given as monotherapy, results from both PDX models showed robust synergistic activity when eribulin and palbociclib were combined. These results suggest a scenario in which both drugs exert effects at the G1/S cell cycle checkpoint in ways that are mechanistically synergistic and that lead to therapeutically favorable outcomes. These preclinical results in PDX models thus support clinical use of the combination of eribulin plus palbociclib in cancer patients.

Antitumor activity of eribulin in combination with palbociclib was studied in Swiss nude mice bearing subcutaneous patient-derived ER⁺/Her2⁻breast tumors. As these drugs exert their effects at different stages of the cell cycle (eribulin at mitosis and palbociclib at the G1/S checkpoint), a dosing regimen in which the drugs are administered in a staggered manner was used, as explained below.

### Materials and Methods

Eribulin mesylate API was provided in the form of a dry powder and DMSO stocks were prepared and stored at −20° C. or lower. Before injection into mice, eribulin was diluted in 0.9% NaCl at 0.1 mg/mL. Palbociclib was diluted in 0.9% NaCl at 0.1 mg/mL. The drugs were administered in dose volumes of 10 mL/kg/administration, according to the most recent body weight of the mice. Eribulin was administered intravenously (IV, bolus) by injection into the caudal vein of the mice, while palbociclib was administered by oral gavage (per as, PO) using a gavage tube.

The patient-derived breast tumors used in the experiments described in this example are as follows: (i) OD-BRE-0192, an ER⁺, PR⁺, Her2⁻luminal B invasive lobular carcinoma, passage 11; and (ii) OD-BRE-0745, an ER⁺, PR⁺, Her2⁻luminal B infiltrating ductal adenocarcinorna, passage 5; both supplied by Oncodesign Biotechnology (France).

### Induction of Breast Tumors in Nude Mice

Patient-derived breast cancer fragments were subcutaneously implanted into the right flank of thirty (30) female Swiss nude mice per model. When tumor volumes reached 500-1000 mm³, tumors were surgically excised and tumor fragments (30-50 mg) were subcutaneously implanted into the right flanks of 101 female Swiss nude mice per model. All fragment implantations were performed 24 to 72 hours after whole body irradiation with a γ-source (2 Gy, ⁶⁰Co, BioMEP, France).

### Treatment Schedule

A dosing scheme was devised in order to avoid possible cell cycle-based antagonism. In particular, previous cell-based in vitro studies examining combinations of eribulin and palbociclib suggested the possibility that simultaneous exposure to the two drugs could result in cell cycle-based antagonism, in which the antimitotic activity or eribulin prevented cells from reaching the G1/S cell cycle checkpoint where palbociclib exerts its CDK 4/6 inhibitory activity, and the CDK 4/6 inhibitory activity of palbociclib at the G1/S checkpoint prevented cells from reaching mitosis where eribulin exerts its antimitotic activity. To prevent such antagonism in the in vivo PDX studies, a ‘palbociclib holiday’ scheme was used in which palbociclib was not given the day before or the day of eribulin administration. In this way, eribulin was not administered less than 48 hours after the last palbociclib dosing, allowing sufficient time for G1/S cell cycle blockage by palbociclib to recover. The asymmetric nature of the holiday (48-hours recovery from the G1/S cell cycle block of palbociclib before eribulin versus 24-hours recovery from the antimitotic effects eribulin before palbociclib) was based on the presumption that re-entering the cell cycle from a G1/S block (essentially G0) would inherently be a slower process than resumption and completion of mitosis after levels of eribulin had dropped below threshold levels required to induce mitotic blocks.

For each model, the treatment started when the tumors reached a mean volume of 200-300 mm³. The day of randomization was considered as D0. Seventy two (72) animals out of one hundred and one (101) were randomized according to their individual tumor volume into 9 groups, each of 8 animals, using Vivo manager® software (Biosystemes, Couternon, France). A statistical test (analysis of variance) was performed to test for homogeneity between groups. The treatment with eribulin started the day of randomization (D0) and the treatment with palbociclib started one day after the randomization (D1). The treatment schedules used for the OD-BRE-0192 model are as follows:

The mice from group 1 received three IV injections of eribulin vehicle once per day every 7 days (at D0, D7, and D14: Q7D×3), in combination with 3 cycles of one daily PO administration of palbociclib vehicle for 5 consecutive days, with each palbociclib vehicle cycle separated by a 2-day period of wash out (from D1 to D5, D8 to D12, and D15 to D19: (Q1D×5)×3W),

The mice from group 2 received three IV injections of eribulin at 0.1 mg/kg once per day every 7 days (at D0, D7, and D14: Q7D×3),

The mice from group 3 received three IV injections of eribulin at 0.25 mg/kg once per day every 7 days (at D0, D7, and D14: Q7D×3),

The mice from group 4 received 3 cycles of one daily PO administration of palbociclib at 75 mg/kg for 5 consecutive days, with each palbociclib cycle separated by a 2-day period of wash out (from D1 to D5, D8 to D12, and D15 to D19: (Q1D×5)×3W),

The mice from group 5 received 3 cycles of one daily PO administration of palbociclib at 150 mg/kg for 5 consecutive days, with each palbociclib cycle separated by a 2-day period of wash out (from D1 to D5, D8 to D12, and D15 to D19: (Q1D×5)×3W),

The mice from group 6 received three IV injections of eribulin at 0.1 mg/kg once per day every 7 days (at D0, D7, and D14: Q7D×3), in combination with 3 cycles of one daily PO administration of palbociclib at 75 mg/kg for 5 consecutive days, with each palbociclib cycle separated by a 2-day period of wash out (from D1 to D5, D8 to D12, and D15 to D19: (Q1D×5)×3W),

The mice from group 7 received three IV injections of eribulin at 0.1 mg/kg once per day every 7 days (at D0, D7, and D14: Q7D×3), in combination with 3 cycles of one daily PO administration of palbociclib at 150 mg/kg for 5 consecutive days, with each palbociclib cycle separated by a 2-day period of wash out (from D1 to D5, D8 to D12, and D15 to D19: (Q1D×5)×3W),

The mice from group 8 received three IV injections of eribulin at 0.25 mg/kg once per day every 7 days (at D0, D7, and D14: Q7D×3), in combination with 3 cycles of one daily PO administration of palbociclib at 75 mg/kg for 5 consecutive days, with each palbociclib cycle separated by a 2-day period of wash out (from D1 to D5, D8 to D12, and D15 to D19: (Q1D×5)×3W),

The mice from group 9 received three IV injections of eribulin at 0.25 mg/kg once per day every 7 days (at D0, D7, and D14: Q7D×3), in combination with 3 cycles of one daily PO administration of palbociclib at 150 mg/kg for 5 consecutive days, with each palbociclib cycle separated by a 2-day period of wash out (from D1 to D5, D8 to D12, and D15 to D19: (Q1D×5)×3W).

The treatment schedules for the OD-BRE-0192 model are summarized in the following table:

| | No. | | Dose | Administration | Treatment |
|---|---|---|---|---|---|
| Group | Animals | Treatment | (mg/kg) | Route | Schedule* |
| 1 | 8 | Eribulin | — | IV | Q7Dx3 |
| | | vehicle | | | |
| | | Palbociclib | — | PO | (Q1Dx5)x3W |
| | | vehicle | | | |
| 2 | 8 | Eribulin | 0.1 | IV | Q7Dx3 |
| 3 | 8 | Eribulin | 0.25 | IV | Q7Dx3 |
| 4 | 8 | Palbociclib | 75 | PO | (Q1Dx5)x3W |
| 5 | 8 | Palbociclib | 150 | PO | (Q1Dx5)x3W |
| 6 | 8 | Eribulin | 0.1 | IV | Q7Dx3 |
| | | Palbociclib | 75 | PO | (Q1Dx5)x3W |
| 7 | 8 | Eribulin | 0.1 | IV | Q7Dx3 |
| | | Palbociclib | 150 | PO | (Q1Dx5)x3W |
| 8 | 8 | Eribulin | 0.25 | IV | Q7Dx3 |
| | | Palbociclib | 75 | PO | (Q1Dx5)x3W |
| 9 | 8 | Eribulin | 0.25 | IV | Q7Dx3 |
| | | Palbociclib | 150 | PO | (Q1Dx5)x3W |

*Eribulin or eribulin vehicle was dosed on days 0, 7, and 14, and palbociclib or palbociclib vehicle were dosed on days 1-5, 8-12, and 15-19.

For the OD-BRE-0745 model, the three cycles described above for the OD-BRE-0192 model were carried out. In addition, a fourth cycle was carried out, starting with eribulin administration after a 13-day period of eribulin wash out. For this fourth cycle, eribulin was administered on day 28 and palbociclib was administered on days 29-33.

All study data, including animal body weight measurements, tumor volume, clinical and mortality records, and treatment, were recorded on Vivo Manager® database (Biosystemes, Dijon, France). Viability and behavior were recorded every day. Body weights were measured twice per week. Lengths and widths of tumors were measured twice per week with calipers and the volumes of the tumors were estimated as follows: tumor volume=(width²×length)/2.

### Results

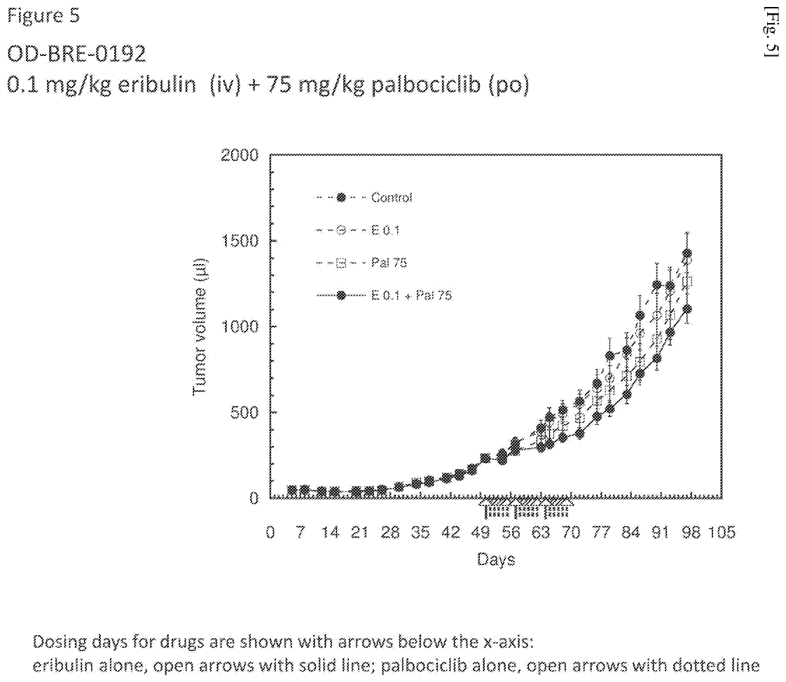

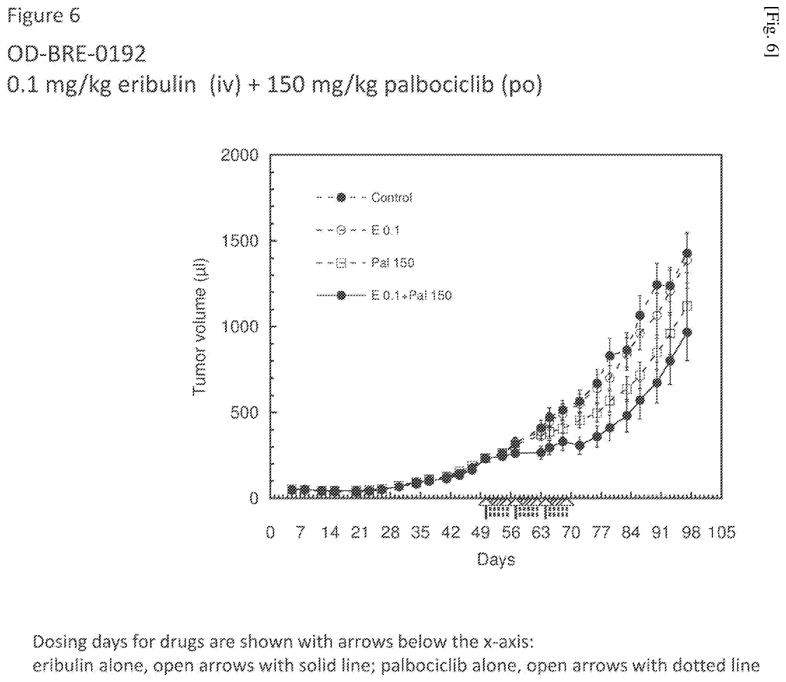

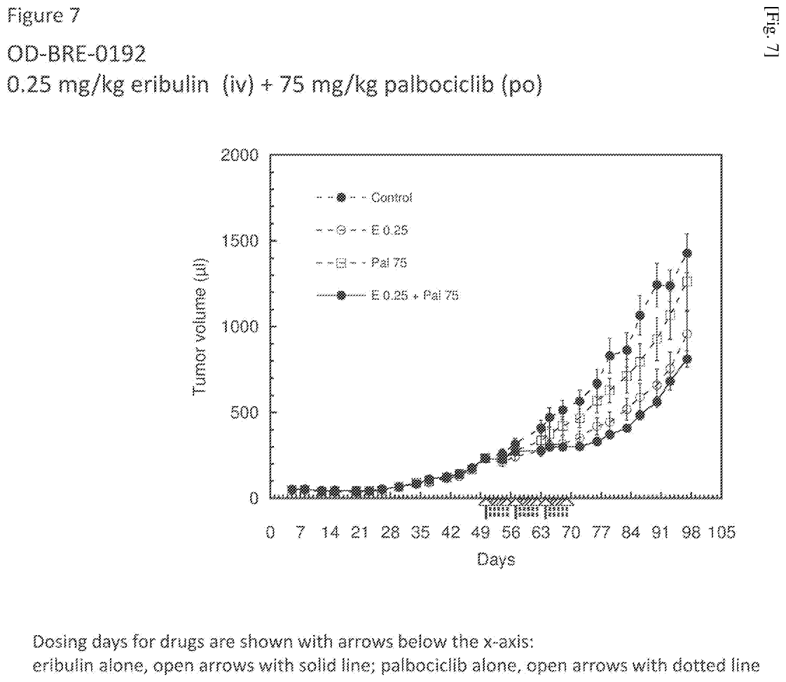

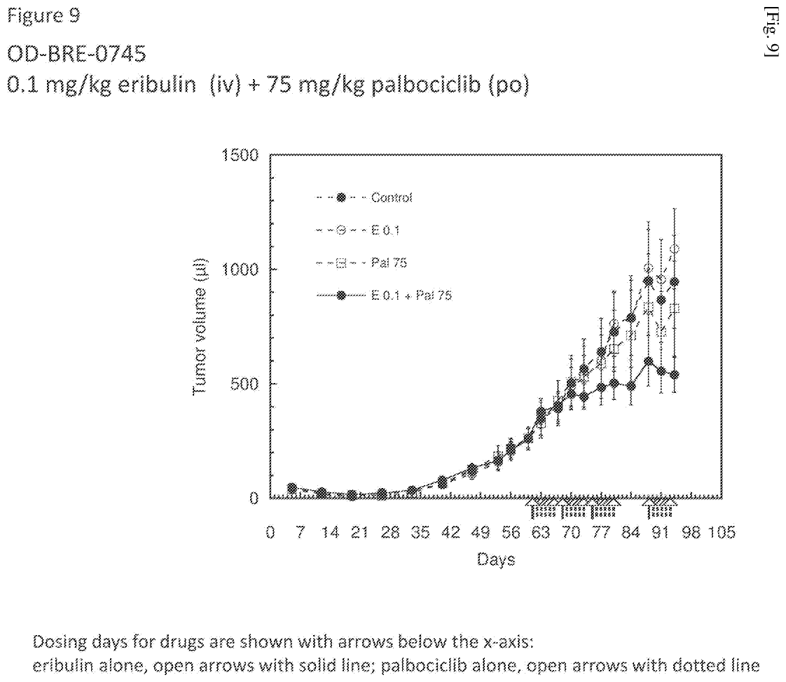

The results of the combination studies are shown in FIGS. 5-8 (OD-BRE-0192) and FIGS. 9-12 (OD-BRE-0745). The day numbers indicated along the x-axes are the days after tumor implantation. Thus, day 0 as indicated on the x-axes of FIGS. 5-12 is the day of tumor implantation. Dosing days are as indicated with coded arrows under the x-axes. Dosing began on day 50 relative to tumor implantation for the OD-BRE-0192 model, and on day 61 relative to tumor implantation for the OD-BRE-0745 model.

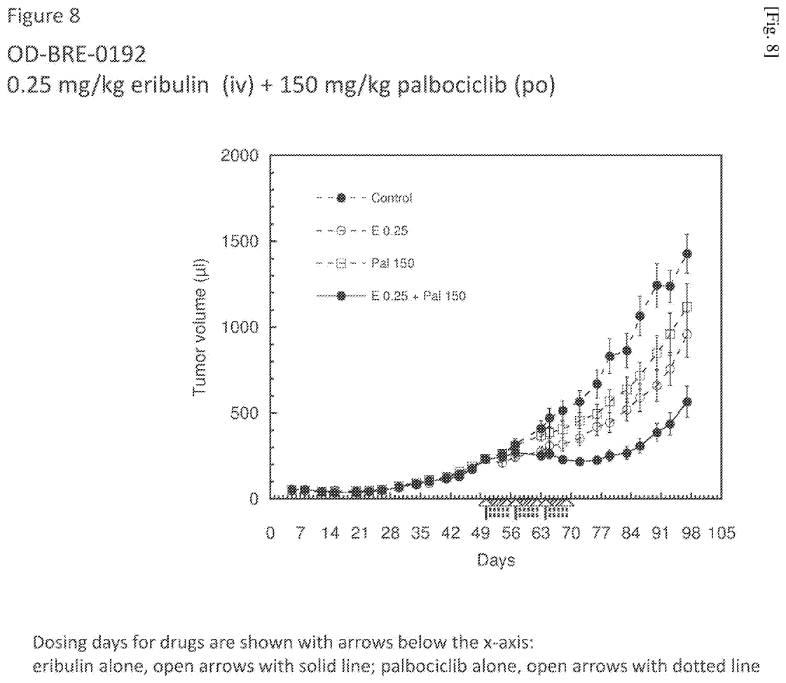

As shown in FIGS. 5-8, for the OD-BRE-0192 model, improved results were obtained with the combination of eribulin and palbociclib, as compared to vehicle only controls and either drug alone. The combination effect is most readily apparent with the higher drug amounts tested (0.25 mg/kg eribulin and 150 mg/kg palbociclib) (see FIG. 8).

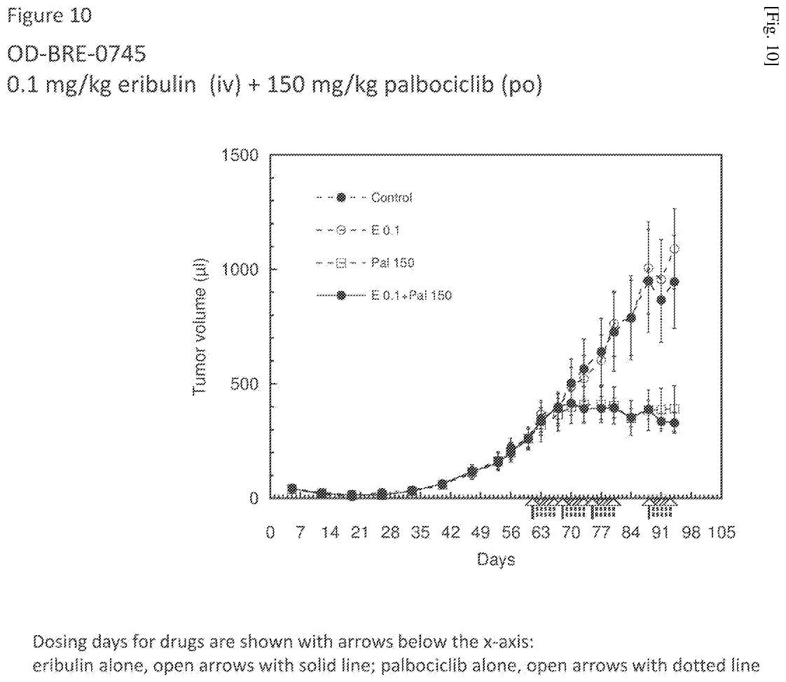

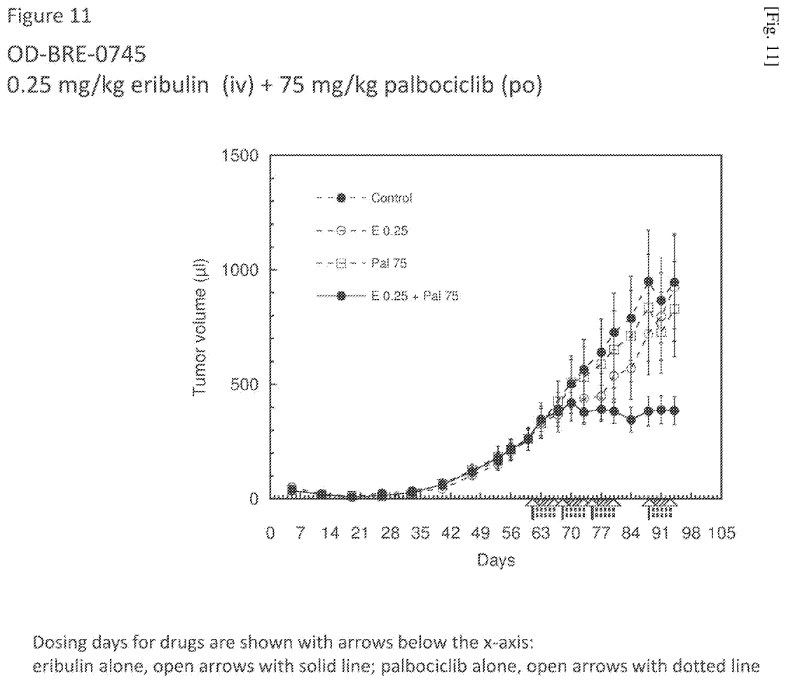

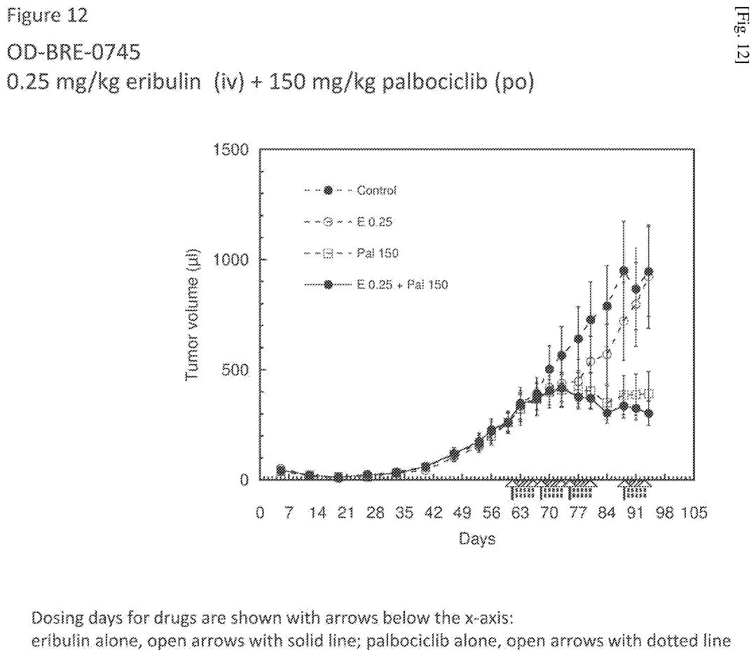

Similarly, as shown in FIGS. 9-12, for the OD-BRE-0745 model, improved results were obtained with the combination of eribulin and palbociclib, as compared to vehicle only controls and either drug alone. At the higher amount of palbociclib tested (150 mg/kg palbociclib), however, palbociclib overwhelmed the response and the contribution of eribulin is masked (FIGS. 10 and 12). The combination effect is thus more readily apparent when the lower amount of palbociclib (75 mg/kg) was used (see FIGS. 9 and 11).

Eribulin and palbociclib showed, synergistic anticancer activity in two PDX models of ER+/PR+/Her2−luminal B human breast cancer. All doses and combinations were at or below empirically determined MTD dose levels (based on standard criteria of <20% reversible body weight loss and <10% lethality). In both models, synergy was seen with doses intentionally selected to show only minimal anticancer activity when administered as single agents. A 48-hour ‘palbociclib holiday’ dosing strategy was employed to avoid potential cell cycle-based antagonism. In the OD-BRE-0192 PDX model, synergy was optimally seen with 0.25 mg/kg eribulin plus 150 mg/kg palbociclib. In the OD-BRE-0745 PDX model, synergy was optimally seen with 0.25 mg/kg eribulin plus 75 mg/kg palbociclib.

Under these conditions described above, combining eribulin and palbociclib led to markedly superior anticancer activity in both models (minimum T/C values of 29% and 41%) compared to either agent alone (T/C: 55-67% and 88-98%, respectively). These preclinical PDX results thus support clinical use of eribulin and palbociclib combinations for, e.g., appropriate patients with ER+/Her2−breast cancers.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form. Other embodiments are within the scope of the following claims.

1. A method for treating a subject having or at risk of developing cancer, the method comprising administering to the subject (a) eribulin, or a pharmaceutically acceptable salt thereof, and (b) a cyclin dependent kinase (CDK) inhibitor.

2. The method of claim 1, wherein the pharmaceutically acceptable salt of eribulin is eribulin mesylate.

3. The method of claim 1 or 2, wherein the CDK inhibitor is a CDK 4 inhibitor, a CDK 6 inhibitor, or a CDK 4/6 inhibitor.

4. The method of any one of claims 1-3, wherein the CDK inhibitor is selected from the group consisting of palbociclib, ribociclib, G1T-28, abemaciclib, and MM-D37K.

5. The method of any one of claims 1-4, wherein the CDK inhibitor is palbociclib.

6. The method of any one of claims 1-5, wherein the method consists of administering to the subject (a) eribulin mesylate and (b) the CDK inhibitor.

7. The method of any one of claims 1-6, wherein the method consists of administering to the subject (a) eribulin mesylate and (b) palbociclib.

8. The method of any one of claims 1-7, wherein (b) is withheld for a certain period of time during said regimen.

9. The method of claim 8, wherein (b) is withheld for one or more days before, during, or after (a) is administered.

10. The method of claim 9, wherein (b) is withheld for two days before, during, or after (a) is administered.

11. The method of any one of claims 1-10, wherein (b) is not administered within about 24-48 hours before (a).

12. The method of any one of claims 1-11, wherein (b) is not administered within about 24 hours after (a).

13. The method of any one of claims 1-12, wherein (a) is administered on days 1 and 8 of a 21 day cycle.

14. The method of claim 13, wherein (b) is administered on any one or more of days 2-6 (or 7) and 9-13 (or 14) of said 21 day cycle.

15. The method of any one of claims 1-12, wherein (a) is administered on days 1, 8 and 15 of a 28 day cycle.

16. The method of 15, wherein (b) is administered on any one or more of days 2-6 (or 7), 9-13 (or 14) and 16-20 (or 21) of said 28 day cycle.

17. The method of any one of claims 1-16, wherein the subject is a human.

18. The method of any one of claims 1-17, wherein the subject is diagnosed with cancer, in treatment for cancer, or in post-therapy recovery from cancer.

19. The method of any one of claims 1-18, wherein the cancer is a primary tumor, is locally advanced, or is metastatic.

20. The method of any one of claims 1-19, wherein the cancer is selected from the group consisting of breast cancer, sarcomas, endometrial cancer, ovarian cancer, prostate cancer, leukemia, lymphoma, lung cancer, neuroendocrine tumors, pheochromocytoma, and thyroid cancer.

21. The method of claim 20, wherein the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

22. The method of any one of claims 1-21, wherein the cancer is a hormone responsive cancer.

23. The method of any one of claims 1-22, wherein said subject is an adult patient.

24. The method of any one of claims 1-22, wherein said subject is a pediatric patient.

25. The method of any one of claims 1-24, wherein the eribulin or the pharmaceutically acceptable salt thereof is administered by intravenous infusion.

26. The method of claim 25, wherein the intravenous infusion is for about 1 to about 20 minutes.

27. The method of claim 26, wherein the intravenous infusion is for about 2 to about 5 minutes.

28. The method of any one of claims 1-27, wherein the eribulin or the pharmaceutically acceptable salt thereof is administered in an amount in the range of about 0.1 mg/m² to about 20 mg/m².

29. The method of claim 28, wherein the eribulin or the pharmaceutically acceptable salt thereof is administered in an amount of about 1.1 mg/m² or 1.4 mg/m².

30. The method of any one of claims 1-29, wherein the CDK inhibitor is administered orally in an amount ranging from 5-350 mg once or twice per day.

31. The method of claim 30, wherein the CDK inhibitor is palbociclib and is administered in an amount of about 125 mg, 100 mg, 75 mg, 50 mg, or 25 mg per dose.

32. The method of any one of claims 1-31, wherein the treating: (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

33. A method for decreasing the size of a tumor in a subject, the method comprising administering to the subject (a) eribulin, or a pharmaceutically acceptable salt thereof, and (b) a CDK inhibitor.

34. The method of claim 33, wherein the pharmaceutically acceptable salt of eribulin is eribulin mesylate.

35. The method of claim 33 or 34, wherein the CDK inhibitor is palbociclib.

36. The method of any one of claims 1 to 35, further comprising administration of one or more additional therapeutic agents.

37. The method of claim 36, wherein the one or more additional therapeutic agents is an anti-hormonal agent.

38. The method of claim 37, wherein the anti-hormonal agent is selected from fulvestrant, tamoxifen, toremifene, and an aromatase inhibitors, such as letrozole.

39. The method of claim 36, wherein the one or more additional therapeutic agents is selected from an immunomodulatory agent, a chemotherapeutic/antitumor agent, an antibacterial agent, an anti-emetic, and, and an anti-inflammatory agent.

40. The method of claim 39, wherein the immunomodulatory agent is an antibody or a vaccine.

41. A kit for use in treating cancer or decreasing tumor size in a subject, the kit comprising (a) eribulin, or a pharmaceutically acceptable salt thereof, and (b) a CDK inhibitor, optionally in dosage form.

42. The kit of claim 41, wherein the pharmaceutically acceptable salt of eribulin is eribulin mesylate.

43. The kit of claim 41 or 42, wherein the CDK inhibitor is palbociclib, ribociclib, G1T-28, abemaciclib, or MM-D37K.

44. Eribulin, or a pharmaceutically acceptable salt thereof, for use in a method for treating a subject having or at risk of developing cancer, the method comprising administering to the subject (a) eribulin, or pharmaceutically acceptable salt thereof, and (b) a CDK inhibitor.

45. Eribulin, or a pharmaceutically acceptable salt thereof, for use in a method of making a medicament for treating a subject having or at risk of developing cancer, the method comprising administering to the subject (a) eribulin, or pharmaceutically acceptable salt thereof, and (b) a CDK inhibitor.

46. The use of claim 44 or 45, further comprising administration of one or more additional therapeutic agents.

47. The use of claim 46, wherein the one or more additional therapeutic agents is an anti-hormonal agent.

48. The use of claim 47, wherein the anti-hormonal agent is selected from fulvestrant, tamoxifen, toremifene, and an aromatase inhibitors, such as letrozole.

49. The use of claim 46, wherein the one or more additional therapeutic agents is selected from an immunomodulatory agent, a chemotherapeutic/antitumor agent, an antibacterial agent, an anti-emetic, and, and an anti-inflammatory agent.

50. The use of claim 49, wherein the immunomodulatory agent is an antibody or a vaccine.
